(19) United States
(12) Patent Application Publication
(10) Pub. No.: US 2010/0069312 A1
(43) Pub. Date: Mar. 18, 2010

(54) AMINOALKYL GLUCOSAMINE PHOSPHATE COMPOUNDS FOR TREATING AUTOIMMUNE DISEASES

(75) Inventors: Stanislas Goriely, Bruxelles (BE); Dominique De Wit, Anderlecht (BE); Michel Goldman, Uccle (BE); Sophie Detienne, Luttre (BE); Jean-Marc Brulet, Mons (BE)

Correspondence Address: KNOBBE MARTENS OLSON & BEAR LLP, 2040 MAIN STREET, FOURTEENTH FLOOR, IRVINE, CA, 92614, US

(73) Assignee: Universite Libre de Bruxelles, Bruxelles (BE)

(21) Appl. No.: 12/595,342

(22) PCT Filed: Apr. 18, 2008
(86) PCT No.: PCT/EP08/54738

§ 371 Date: Oct. 9, 2009

(30) Foreign Application Priority Data
Apr. 19, 2007 (EP) 07106520.5

(51) Int. Cl. A61K 31/7028; C07H 15/04; A61P 37/00

(52) U.S. Cl. 514/25; 536/17.1

(57) ABSTRACT

The invention provides prophylactic and therapeutic applications of select aminoalkyl glucosamine phosphate (AGP) compounds in autoimmune diseases.

### FIELD OF THE INVENTION

The invention relates to prophylactic and therapeutic applications of select aminoalkyl glucosamine phosphate (AGP) compounds in autoimmune diseases.

### BACKGROUND OF THE INVENTION

Synthetic mono- and disaccharide mimetics of monophosphoryl lipid A (MLPA), referred to as aminoalkyl glucosamine phosphate (AGP) compounds, have been described previously as adjuvants in vaccine formulations, i.e., for augmenting antibody protection in immunised subjects (see, e.g., WO 98/50399 A1).

More recently, U.S. Pat. No. 6,800,613 B2, US 2003/0105032 A1 and US 2004/0147480 A1 have suggested to use the broad class of AGP compounds as monotherapies, i.e., without an exogenous antigen, in the prevention and treatment of infections diseases, autoimmunity and allergies. In particular, the latter documents teach that the treatment of autoimmune diseases requires AGP compounds which are capable of antagonising or inhibiting one or more Toll-like receptors, particularly TLR-2 and/or TLR-4 receptors, whereby an autoimmune response is ameliorated (see col. 6, l. 37-46 of U.S. Pat. No. 6,800,613 B2, par. [0044] of US 2003/0105032 A1 and par. [0041] of US 2004/0147480 A1).

In view of the very broad, generic disclosure of the above documents, there exists a considerable need in the art to identify AGP compounds particularly useful in the prevention and treatment of autoimmune diseases, e.g., AGP compounds that are highly therapeutically effective in one or more autoimmune diseases.

### SUMMARY OF THE INVENTION

The present invention addresses the above discussed needs in the art. More specifically, the inventors surprisingly identified a particular subgroup of AGP compounds showing high efficacy in the therapy of autoimmune diseases, and in particular multiple sclerosis (MS).

Accordingly, in an aspect, the invention provides a compound having the formula (I):

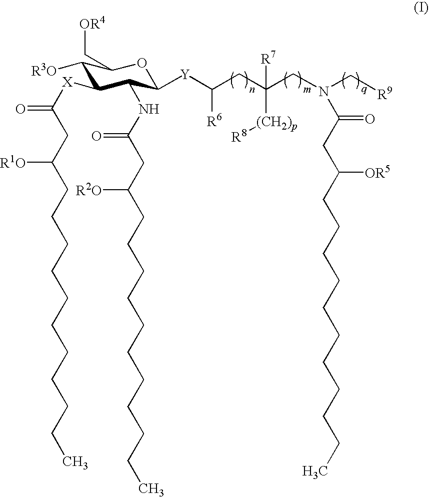

and pharmaceutically acceptable salts thereof, wherein:
X is —O— or —NH—; Y is —O— or —S—; the subscripts n, m, p and q are each independently an integer of from 0 to 6; R¹, R² and R⁵ are each independently a (C₈-C₁₄)acyl group and at least one of R¹, R² and R⁵ is a (C₁₀)acyl group; R³ is —H or —PO₃R¹¹R¹², wherein R¹¹ and R¹² are each independently —H or (C₁-C₄)alkyl; R⁴ is —H, —CH₃ or PO₃R¹³R¹⁴, wherein R¹³ and R¹⁴ are each independently —H or (C₁-C₄)alkyl, with the proviso that when R³ is —PO₃R¹¹R¹², R⁴ is other than —PO₃R¹³R¹⁴; R⁶ and R⁷ are each independently —H or —CH₃; and R⁸ and R⁹ are each independently —H, —OH, (C₁-C₄)alkoxy, —PO₃R¹⁵R¹⁶, —OPO₃R¹⁵R¹⁶, —SO₃R¹⁵, —OSO₃R¹⁵, —NR¹⁵R¹⁶, —SR¹⁵, —C≡N, —NO₂, —C(═O)H, —C(═O)OR¹⁵, or —C(═O)NR¹⁵R¹⁶, wherein R¹⁵ and R¹⁶ are each independently —H or (C₁-C₄)alkyl;

for use in the treatment, e.g., prophylactic and/or therapeutic treatment, of an autoimmune disease.

The invention also provides a pharmaceutical composition comprising the compound having the formula (I) or pharmaceutically acceptable salt thereof as defined above for use in the treatment, e.g., prophylactic and/or therapeutic treatment, of an autoimmune disease.

The invention as well provides the use of the compound having the formula (I) or pharmaceutically acceptable salt thereof as defined above for the manufacture of a medicament for the treatment, e.g., prophylactic and/or therapeutic treatment, of an autoimmune disease.

Also, the invention provides a method for treating, e.g., prophylactically and/or therapeutically treating, an autoimmune disease in a subject in need of said treatment, comprising administering to said subject a therapeutically effective amount of the compound having the formula (I) or pharmaceutically acceptable salt thereof as defined above.

As noted, at least one of the substituents R¹, R² and R⁵ of the compound having the formula (I) or its pharmaceutically acceptable salts as defined above is a (C₁₀)acyl group. Preferably, at least two of said substituents R¹, R² and R⁵ may be a (C₁₀)acyl group, and even more preferably all three said substituents R¹, R² and R⁵ may be a (C₁₀)acyl group.

It was known in the art that AGP compounds comprising a (C₁₀)acyl group as one or more of the substituents R¹, R² and R⁵ may be particularly potent agonists of Toll-like receptors, especially of the TLR4 receptor (see, e.g., Stöver et al. 2004. JBC 279: 4440-4449). Therefore, it was entirely unexpected and surprising—in view of the above detailed teachings of U.S. Pat. No. 6,800,613 B2, US 2003/0105032 A1 and US 2004/0147480 A1 that autoimmune diseases need to be treated using AGP compounds which antagonise or inhibit the TLR4 receptor—that the herein specified AGP compounds should be so therapeutically effective in autoimmune diseases, and particularly in multiple sclerosis.

In preferred, non-limiting embodiments, the invention may use the compound having the formula (I) and pharmaceutically acceptable salts thereof as defined above, wherein X is —O—; and/or Y is —O—; and/or the subscripts n, m, p, q are each independently integers from 0 to 3, preferably from 0 to 2, more preferably from 0 to 1, and most preferably 0; and/or R³ is —PO₃H₂; and/or R⁴ is —H; and/or R⁶ is —H; and/or R⁷ is —H; and/or R⁸ is —C(═O)OH and/or R⁹ is —H.

In a particularly preferred embodiment, the invention may use the compound having the formula (I) and pharmaceutically acceptable salts thereof as defined above, wherein X is —O—; Y is —O—; the subscripts n, m, p, q are each 0; R³ is —PO₃H₂; R⁴, R⁶, R⁷ and R⁹ are each —H; and R⁸ is —C(═O)OH.

Even more preferably, the invention may employ the compound having the formula (II):

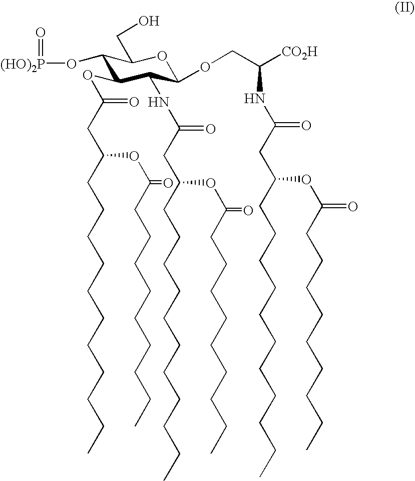

and pharmaceutically acceptable salts thereof.

In another particularly preferred embodiment of the invention, the autoimmune disease to be treated using the above compounds and pharmaceutically acceptable salts, or using pharmaceutical compositions comprising such, is multiple sclerosis (MS).

These and further aspects and preferred embodiments of the invention are described in the following sections and in the appended claims.

### BRIEF DESCRIPTION OF FIGURES

FIG. 1 illustrates the effect of compound formula (II) treatment on IL-17 production by lymph node cells of PLP₁₃₉₋₁₅₁ immunised mice. Grey bar: no treatment, black bar: treatment with 2.5 μg/mouse of compound formula (II) (p<0.05).

### DETAILED DESCRIPTION OF THE INVENTION

As used herein, the singular forms “a”, “an”, and “the” include both singular and plural referents unless the context clearly dictates otherwise. By way of example, “a fibre” refers to one or more than one fibres.

The terms “comprising”, “comprises” and “comprised of” as used herein are synonymous with “including”, “includes” or “containing”, “contains”, and are inclusive or open-ended and do not exclude additional, non-recited members, elements or method steps.

The recitation of numerical ranges by endpoints includes all numbers and fractions subsumed within that range, as well as the recited endpoints.

The term “about” as used herein when referring to a measurable value such as a parameter, an amount, a temporal duration, and the like, is meant to encompass variations of ±20% or less, preferably ±10% or less, more preferably ±5% or less, even more preferably ±1% or less, and still more preferably ±0.1% or less from the specified value, insofar such variations are appropriate to perform in the disclosed invention. It is to be understood that the value to which the modifier “about” refers is itself also specifically, and preferably, disclosed.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention. When specific terms are defined in connection with a particular aspect or embodiment, such connotation is meant to apply throughout this specification, i.e., also in the context of other aspects or embodiments, unless otherwise defined.

Compounds

The Summary section specifies aspects of the invention involving prophylactic and therapeutic applications of the above defined compound having the formula (I) and pharmaceutically acceptable salts thereof in autoimmune disorders.

As used herein, “—” when in between two atoms, indicates a singe bond between the said atoms; “═” when in between two atoms, indicates a double bond between the said atoms; “≡” when in between two atoms, indicates a triple bond between the said atoms.

As used herein, “—” when projecting from an atom of a substituting radical, indicates point(s) of attachment of the said substituting radical to the atom being substituted with the said radical.

The term “substituted” as used herein means that one or more hydrogens on the atom (typically a C-, N-, O- or S-atom, usually a C-atom) indicated by the modifier “substituted” is replaced with a selection from the specified group, provided that the indicated atom's normal valence is not exceeded, and that the substitution results in a chemically stable compound, i.e., a compound that is sufficiently robust to survive preparation and/or isolation to a useful degree of purity. The term “one or more” covers the possibility of all the available atoms, where appropriate, to be substituted, preferably, one, two or three. When any variable, e.g., halogen or alkyl, occurs more than one time in any constituent, each definition is independent.

The term “alkyl”, as used herein alone or as part of another group, means a straight, branched or cyclic, or a combination of straight and cyclic or branched and cyclic, monovalent hydrocarbon radical, which may be fully saturated, mono- or poly-unsaturated or aromatic, and includes the designated number of carbon atoms, e.g., (C₁-C₄)alkyl denotes an alkyl radical having between 1 and 4 carbon atoms, e.g., 1, 2, 3 or 4 carbon atoms. Examples of saturated alkyl radicals include, without limitation, groups such as methyl, ethyl, n-propyl, isopropyl, n-butyl, isobutyl, sec-butyl, tert-butyl, cyclohexyl, cyclohexylmethyl, cyclopropylmethyl, homologues and isomers of, e.g., n-pentyl, n-hexyl, n-heptyl, n-octyl, and the like. An unsaturated alkyl group is one having one or more double bonds and/or triple bonds, preferably one or more double bonds. Examples of unsaturated alkyl radicals include, without limitation, groups such as ethenyl, methylethenyl, propenyl, 1-butenyl, 2-butenyl, 2-butadienyl, 2,4-pentadienyl, ethynyl, 1- and 3-propynyl, 3-butynyl and the higher homologues and isomers. The term “(C₁-C₄)alkyl” also specifically encompasses, e.g., (C₁-C₂)alkyl, (C₁-C₃)alkyl, (C₂-C₃)alkyl, (C₂-C₄)alkyl and (C₃-C₄)alkyl.

The term “alkoxy” or “alkyloxy” as used herein alone or as part of another group, means an alkyl ether radical, wherein the term alkyl is as defined above. Accordingly, the term is meant to include groups otherwise defined as —O-alkyl.

The term “acyl” as used herein alone or as part of another group, refers to a group derived from an organic acid by removal of the hydroxy (—OH) group. Accordingly, the term is meant to include groups otherwise defined as —C(═O)-alkyl, wherein the term alkyl is as defined above.

Hence, (C₈)acyl includes the group —C(═O)—(C₇)alkyl, (C₉)acyl includes the group —C(═O)—(C₈)alkyl, (C₁₀)acyl includes the group —C(═O)—(C₉)alkyl, (C₁₁)acyl includes the group —C(═O)—(C₁₀)alkyl, (C₁₂)acyl includes the group —C(═O)—(C₁₁)alkyl, (C₁₃)acyl includes the group —C(═O)—(C₁₂)alkyl, and (C₁₄)acyl includes the group —C(═O)—(C₁₃)alkyl.

The term “(C₈-C₁₄)acyl” also specifically encompasses (C₈-C₉)acyl, (C₈-C₁₀)acyl, (C₈-C₁₁)acyl, (C₈-C₁₂)acyl, (C₈-C₁₃)acyl, (C₉-C₁₀)acyl, (C₉-C₁₁)acyl, (C₉-C₁₂)acyl, (C₉-C₁₃)acyl, (C₉-C₁₄)acyl, (C₁₀-C₁₁)acyl, (C₁₀-C₁₂)acyl, (C₁₀-C₁₃)acyl, (C₁₀-C₁₄)acyl, (C₁₁-C₁₂)acyl, (C₁₁-C₁₃)acyl, (C₁₁-C₁₄)acyl, (C₁₂-C₁₃)acyl, (C₁₂-C₁₄)acyl, and (C₁₃-C₁₄)acyl.

Each of the above terms (e.g., “alkyl”, “alkoxy”, “acyl”) are meant to include both un-substituted and substituted forms of the indicated radical. Preferably, substituents for these radicals may be chosen from the group comprising or consisting of: —OR′; ═O; ═NR′; ═N—OR′; —NR′R″; —SR′; -halogen, preferably —F, —Cl, —Br or —I, more preferably —F, —Cl or —Br, even more preferably —F or —Cl; —C(═O)R′; —OC(═O)R′; —C(═O)OR′; —C(═O)NR′R″; —O(C═O)NR′R″; —NR″C(═O)R′; —NR′C(═O)NR′R′″; —NR″C(═O)OR′; —C(═NH)NH₂; —C(═NR′)NH₂; —NH—C(═NH)NH₂; —NR′—C(═NH)NH₂; —NH—C(═NR′)NH₂; —S(═O)R′; —S(═O)OR′; —S(═O)₂OR′; —S(═O)NR′R″; —S(═O)₂NR′R″; —C≡N; and —NO₂; wherein R′, R″ and R′″ are each independently —H or un-substituted (C₁-C₈)alkyl, preferably each independently —H or un-substituted (C₁-C₄)alkyl, more preferably each independently —H or un-substituted (C₁-C₂)alkyl. When R′ and R″ are attached to the same nitrogen atom, they can be combined with the nitrogen atom to form a 5-, 6-, or 7-membered ring.

More preferably, substituents for said radicals (e.g., “alkyl”, “alkoxy”, “acyl”) may be chosen from the group comprising or consisting of: —OR′; ═O; —NR′R″; —SR′; -halogen, preferably —F, —Cl, —Br or —I, more preferably —F, —Cl or —Br, even more preferably —F or —Cl; —C(═O)R′; —OC(═O)R′; —C(═O)OR′; —C(═O)NR′R″; —NR″C(═O)R′; —S(═O)₂OR′; —S(═O)₂NR′R″; —C≡N; and —NO₂; wherein R′ and R″ are as defined above. When R′ and R″ are attached to the same nitrogen atom, they can be combined with the nitrogen atom to form a 5-, 6-, or 7-membered ring.

Even more preferably, substituents for said radicals (e.g., “alkyl”, “alkoxy”, “acyl”) may be chosen from the group comprising or consisting of: —OR′; -halogen, preferably —F, —Cl, —Br or —I, more preferably —F, —Cl or —Br, even more preferably —F or —Cl; —C(═O)R′; —OC(═O)R′; —C(═O)OR′; —S(═O)₂OR′; —C≡N; and —NO₂; wherein R′ is as defined above.

Yet more preferably, substituents for said radicals (e.g., “alkyl”, “alkoxy”, “acyl”) may be chosen from the group comprising or consisting of: —OR′, preferably —OH; and -halogen, preferably —F, —Cl, —Br or —I, more preferably —F, —Cl or —Br, even more preferably —F or —Cl; wherein R′ is are as defined above.

In preferred, albeit non-limiting, embodiments “E1” to “E102”, the compound having the formula (I) and pharmaceutically acceptable salts thereof may include the subscripts and substituents as defined here below.

Embodiment “E1”: X is —O— or —NH—. Embodiment “E2”: X is —O—.

Embodiment “E3”: Y is —O— or —S—; X is as defined in any of “E1” or “E2”. Embodiment “E4”: Y is —O—; X is as defined in any of “E1” or “E2”.

Embodiment “E5”: the subscripts n, m, p and q are each independently an integer of from 0 to 6; X is as defined in any of “E1” or “E2”; Y is as defined in any of “E3” or “E4”. Embodiment “E6”: the subscripts n, m, p and q are each independently an integer of from 0 to 3; X is as defined in any of “E1” or “E2”; Y is as defined in any of “E3” or “E4”. Embodiment “E7”: the subscripts n, m, p and q are each independently an integer of from 0 to 2; X is as defined in any of “E1” or “E2”; Y is as defined in any of “E3” or “E4”. Embodiment “E8”: the subscripts n, m, p and q are each independently 0 or 1; X is as defined in any of “E1” or “E2”; Y is as defined in any of “E3” or “E4”. Embodiment “E9”: the subscripts n, m, p and q are each 0; X is as defined in any of “E1” or “E2”; Y is as defined in any of “E3” or “E4”.

Embodiment “E10”: R¹, R² and R⁵ are each independently a (C₈-C₁₄)acyl group, preferably each independently a (C₈)acyl, (C₁₀)acyl, (C₁₂)acyl or (C₁₄)acyl group, and at least one of R¹, R² and R⁵ is a (C₁₀)acyl group; X is as defined in any of “E1” or “E2”; Y is as defined in any of “E3” or “E4”; the subscripts n, m, p and q are as defined in any of “E5”, “E6”, “E7”, “E8” or “E9”. Embodiment “E11”: R¹, R² and R⁵ are each independently a (C₈-C₁₄)acyl group, preferably each independently a (C₈)acyl, (C₁₀)acyl, (C₁₂)acyl or (C₁₄)acyl group, and at least two of R¹, R² and R⁵ are a (C₁₀)acyl group; X is as defined in any of “E1” or “E2”; Y is as defined in any of “E3” or “E4”; the subscripts n, m, p and q are as defined in any of “E5”, “E6”, “E7”, “E8” or “E9”.

Embodiment “E12”: R¹, R² and R⁵ are each independently a (C₈-C₁₂)acyl group, preferably each independently a (C₈)acyl, (C₁₀)acyl or (C₁₂)acyl group, and at least one of R¹, R² and R⁵ is a (C₁₀)acyl group; X is as defined in any of “E1” or “E2”; Y is as defined in any of “E3” or “E4”; the subscripts n, m, p and q are as defined in any of “E5”, “E6”, “E7”, “E8” or “9”. Embodiment “E13”: R¹, R² and R⁵ are each independently a (C₈-C₁₂)acyl group, preferably each independently a (C₈)acyl, (C₁₀)acyl or (C₁₂)acyl group, and at least two of R¹, R² and R⁵ are a (C₁₀)acyl group; X is as defined in any of “E1” or “E2”; Y is as defined in any of “E3” or “E4”; the subscripts n, m, p and q are as defined in any of “E5”, “E6”, “E7”, “E8” or “9”.

Embodiment “E14”: R¹, R² and R⁵ are each independently a (C₈-C₁₀)acyl group, preferably each independently a (C₈)acyl or (C₁₀)acyl group, and at least one of R¹, R² and R⁵ is a (C₁₀)acyl group; X is as defined in any of “E1” or “E2”; Y is as defined in any of “E3” or “E4”; the subscripts n, m, p and q are as defined in any of “E5”, “E6”, “E7”, “E8” or “E9”.

Embodiment “E15”: R¹, R² and R⁵ are each independently a (C₈-C₁₀)acyl group, preferably each independently a (C₈)acyl or (C₁₀)acyl group, and at least two of R¹, R² and R⁵ are a (C₁₀)acyl group; X is as defined in any of “E1” or “E2”; Y is as defined in any of “E3” or “E4”; the subscripts n, m, p and q are as defined in any of “E5”, “E6”, “E7”, “E8” or “E9”.

Embodiment “E16”: R¹, R² and R⁵ are each a (C₁₀)acyl group; X is as defined in any of “E1” or “E2”; Y is as defined in any of “E3” or “E4”; the subscripts n, m, p and q are as defined in any of “E5”, “E6”, “E7”, “E8” or “E9”.

Preferably, in any of embodiments “E10”, “E11”, “E12”, “E13”, “E14”, “E15” or “E16” at least one, more preferably at least two, and most preferably all three of the acyl groups R¹, R² and R⁵ may be straight, i.e., un-branched.

Also preferably, in any of embodiments “E10”, “E11”, “E12”, “E13”, “E14”, “E15” or “E16” at least one, more preferably at least two, and most preferably all three of the acyl groups R¹, R² and R⁵ may be un-substituted.

Further preferably, in any of embodiments “E10”, “E11”, “E12”, “E13”, “E14”, “E15” or “E16” at least one, more preferably at least two, and most preferably all three of the acyl groups R¹, R² and R⁵ may be saturated. Where any of the acyl groups R¹, R² and R⁵ is unsaturated, said unsaturated acyl group may preferably include ≦6 unsaturated bonds, more preferably ≦4 unsaturated bonds, even more preferably ≦2 unsaturated bonds and most preferably only 1 unsaturated bond. Preferably, ≦2, more preferably ≦1 and most preferably none of the unsaturated bonds in said unsaturated acyl group will be triple bond.

Embodiment “E17”: R³ is —H or —PO₃R¹¹R¹², wherein R¹¹ and R¹² are each independently —H or (C₁-C₄)alkyl; X is as defined in any of “E1” or “E2”; Y is as defined in any of “E3” or “E4”; the subscripts n, m, p and q are as defined in any of “E5”, “E6”, “E7”, “E8” or “E9”; and R¹, R² and R⁵ are as defined in any of “E10”, “E11”, “E12”, “E13”, “E14”, “E15” or “E16”.

Embodiment “E18”: R³ is —PO₃R¹¹R¹², wherein R¹¹ and R¹² are each independently —H or (C₁-C₄)alkyl; X is as defined in any of “E1” or “E2”; Y is as defined in any of “E3” or “E4”; the subscripts n, m, p and q are as defined in any of “E5”, “E6”, “E7”, “E8” or “E9”; and R¹, R² and R⁵ are as defined in any of “E10”, “E11”, “E12”, “E13”, “E14”, “E15” or “E16”.

Embodiment “E19”: R³ is —PO₃H₂; X is as defined in any of “E1” or “E2”; Y is as defined in any of “E3” or “E4”; the subscripts n, m, p and q are as defined in any of “E5”, “E6”, “E7”, “E8” or “E9”; and R¹, R² and R⁵ are as defined in any of “E10”, “E11”, “E12”, “E13”, “E14”, “E15” or “E16”.

Embodiment “E20”: R⁴ is —H, —CH₃ or PO₃R¹³R¹⁴, wherein R¹³ and R¹⁴ are each independently —H or (C₁-C₄)alkyl, with the proviso that when R³ is —PO₃R¹¹R¹², R⁴ is other than —PO₃R¹³R¹⁴; X is as defined in any of “E1” or “E2”; Y is as defined in any of “E3” or “E4”; the subscripts n, m, p and q are as defined in any of “E5”, “E6”, “E7”, “E8” or “E9”; R¹, R² and R⁵ are as defined in any of “E10”, “E11”, “E12”, “E13”, “E14”, “E15” or “E16”; and R³ is as defined in any of “E17”, “E18” or “E19”.

Embodiment “E21”: R⁴ is —H; X is as defined in any of “E1” or “E2”; Y is as defined in any of “E3” or “E4”; the subscripts n, m, p and q are as defined in any of “E5”, “E6”, “E7”, “E8” or “E9”; R¹, R² and R⁵ are as defined in any of “E10”, “E11”, “E12”, “E13”, “E14”, “E15” or “E16”; and R³ is as defined in any of “E17”, “E18” or “E19”.

Embodiment “E22”: R⁶ and R⁷ are each independently —H or —CH₃; X is as defined in any of “E1” or “E2”; Y is as defined in any of “E3” or “E4”; the subscripts n, m, p and q are as defined in any of “E5”, “E6”, “E7”, “E8” or “E9”; R¹, R² and R⁵ are as defined in any of “E10”, “E11”, “E12”, “E13”, “E14”, “E15” or “E16”; R³ is as defined in any of “E17”, “E18” or “E19”; and R⁴ is as defined in any of “E20” or “E21”.

Embodiment “E23”: R⁶ and R⁷ are —H; X is as defined in any of “E1” or “E2”; Y is as defined in any of “E3” or “E4”; the subscripts n, m, p and q are as defined in any of “E5”, “E6”, “E7”, “E8” or “E9”; R¹, R² and R⁵ are as defined in any of “E10”, “E11”, “E12”, “E13”, “E14”, “E15”, or “E16”; R³ is as defined in any of “E17”, “E18” or “E19”; and R⁴ is as defined in any of “E20” or “E21”.

Embodiment “E24”: R⁸ and R⁹ are each independently —H, —OH, (C₁-C₄)alkyloxy, —PO₃R¹⁵R¹⁶, —OPO₃R¹⁵R¹⁶, —SO₃R¹⁵, —OSO₃R¹⁵, —NR¹⁵R¹⁶, —SR¹⁵, —NO₂, —C(═O)H, —C(═O)OR¹⁵, or —C(═O)NR¹⁵R¹⁶, wherein R¹⁵ and R¹⁶ are each independently —H or (C₁-C₄)alkyl; X is as defined in any of “E1” or “E2”; Y is as defined in any of “E3” or “E4”; the subscripts n, m, p and q are as defined in any of “E5”, “E6”, “E7”, “E8” or “E9”; R¹, R² and R⁵ are as defined in any of “E10”, “E11”, “E12”, “E13”, “E14”, “E15” or “E16”; R³ is as defined in any of “E17”, “E18” or “E19”; R⁴ is as defined in any of “E20” or “E21”; and R⁶ and R⁷ are as defined in any of “E22” or “E23”.

Embodiment “E25”: R⁸ and R⁹ are each independently —H, —OH, —OPO₃R¹⁵R¹⁶, —OSO₃R¹⁵, —NO₂, or —C(═O)OR¹⁵, wherein R¹⁵ and R¹⁶ are each independently —H or (C₁-C₄)alkyl; X is as defined in any of “E1” or “E2”; Y is as defined in any of “E3” or “E4”; the subscripts n, m, p and q are as defined in any of “E5”, “E6”, “E7”, “E8” or “E9”; R¹, R² and R⁵ are as defined in any of “E10”, “E11”, “E12”, “E13”, “E14”, “E15” or “E16”; R³ is as defined in any of “E17”, “E18” or “E19”; R⁴ is as defined in any of “E20” or “E21”; and R⁶ and R⁷ are as defined in any of “E22” or “E23”.

Embodiment “E26”: R⁸ and R⁹ are as defined in any of “E24” or “E25” and R⁸ is —C(═O)OR¹⁵, wherein R¹⁵ is —H or (C₁-C₄)alkyl, more preferably R⁸ is —C(═O)OH; X is as defined in any of “E1” or “E2”; Y is as defined in any of “E3” or “E4”; the subscripts n, m, p and q are as defined in any of “E5”, “E6”, “E7”, “E8” or “E9”; R¹, R² and R⁵ are as defined in any of “E10”, “E11”, “E12”, “E13”, “E14”, “E15” or “E16”; R³ is as defined in any of “E17”, “E18” or “E19”; R⁴ is as defined in any of “E20” or “E21”; and R⁶ and R⁷ are as defined in any of “E22” or “E23”.

Embodiment “E27”: R⁸ is —C(═O)OR¹⁵, wherein R¹⁵ is —H or (C₁-C₄)alkyl, more preferably R⁸ is —C(═O)OH, and R⁹ is —H; X is as defined in any of “E1” or “E2”; Y is as defined in any of “E3” or “E4”; the subscripts n, m, p and q are as defined in any of “E5”, “E6”, “E7”, “E8” or “E9”; R¹, R² and R⁵ are as defined in any of “E10”, “E11”, “E12”, “E13”, “E14”, “E15” or “E16”; R³ is as defined in any of “E17”, “E18” or “E19”; R⁴ is as defined in any of “E20” or “E21”; and R⁶ and R⁷ are as defined in any of “E22” or “E23”.

Preferably, in any of embodiments “E17”, “E18”, “E20”, “E24”, “E25”, “E26” or “E27” independently, the recited (C₁-C₄)alkyl group may be a (C₁-C₃)alkyl group, more preferably a C₁ or C₂ alkyl group.

Also preferably, in any of embodiments “E17”, “E18”, “E20”, “E24”, “E25”, “E26” or “E27” independently, the recited (C₁-C₄)alkyl group may be saturated. Where the (C₁-C₄)alkyl group in any of said embodiments is unsaturated, said group may preferably include ≦2 unsaturated bonds, more preferably only 1 unsaturated bond. Preferably, only 1 and more preferably none of said unsaturated bonds will be triple bond.

Further preferably, in any of embodiments “E17”, “E18”, “E20”, “E24”, “E25”, “E26” or “E27” independently, the recited (C₁-C₄)alkyl group may be un-substituted.

It shall be appreciated that the compound having the formula (I) and pharmaceutically acceptable salts thereof may include various combinations of the above defined subscripts and substituents. By means of example and not limitation, Table 1 lists further preferred embodiments of the present compound, wherein X, Y, n, m, p, q, and R¹ to R⁹ are as defined in above embodiments “E1” to “E27”.

TABLE 1
Further preferred examples (embodiments, “Emb.”) of the compound having
the formula (I) and pharmaceutically acceptable salts thereof.

| | X | Y | n, m, p, q | R¹, R², R⁵ | R³ | R⁴ | R⁶, R⁷ | R⁸, R⁹ |
|---|---|---|---|---|---|---|---|---|
| Emb. | as in: | as in: | as in: | as in: | as in: | as in: | as in: | as in: |
| “E28” | “E2” | “E4” | “E8” | “E10” | “E18” | “E21” | “E23” | “E26” |
| “E29” | --//-- | --//-- | --//-- | --//-- | --//-- | --//-- | --//-- | “E27” |
| “E30” | --//-- | --//-- | --//-- | --//-- | “E19” | --//-- | --//-- | “E26” |
| “E31” | --//-- | --//-- | --//-- | --//-- | --//-- | --//-- | --//-- | “E27” |
| “E32” | --//-- | --//-- | “E9” | --//-- | “E18” | --//-- | --//-- | “E26” |
| “E33” | --//-- | --//-- | --//-- | --//-- | --//-- | --//-- | --//-- | “E27” |
| “E34” | --//-- | --//-- | --//-- | --//-- | “E19” | --//-- | --//-- | “E26” |
| “E35” | --//-- | --//-- | --//-- | --//-- | --//-- | --//-- | --//-- | “E27” |
| “E36” | --//-- | --//-- | “E8” | “E11” | “E18” | --//-- | --//-- | “E26” |
| “E37” | --//-- | --//-- | --//-- | --//-- | --//-- | --//-- | --//-- | “E27” |
| “E38” | --//-- | --//-- | --//-- | --//-- | “E19” | --//-- | --//-- | “E26” |
| “E39” | --//-- | --//-- | --//-- | --//-- | --//-- | --//-- | --//-- | “E27” |
| “E40” | --//-- | --//-- | “E9” | --//-- | “E18” | --//-- | --//-- | “E26” |
| “E41” | --//-- | --//-- | --//-- | --//-- | --//-- | --//-- | --//-- | “E27” |
| “E42” | --//-- | --//-- | --//-- | --//-- | “E19” | --//-- | --//-- | “E26” |
| “E43” | --//-- | --//-- | --//-- | --//-- | --//-- | --//-- | --//-- | “E27” |
| “E44” | --//-- | --//-- | “E8” | “E12” | “E18” | --//-- | --//-- | “E26” |
| “E45” | --//-- | --//-- | --//-- | --//-- | --//-- | --//-- | --//-- | “E27” |
| “E46” | --//-- | --//-- | --//-- | --//-- | “E19” | --//-- | --//-- | “E26” |
| “E47” | --//-- | --//-- | --//-- | --//-- | --//-- | --//-- | --//-- | “E27” |
| “E48” | --//-- | --//-- | “E9” | --//-- | “E18” | --//-- | --//-- | “E26” |
| “E49” | --//-- | --//-- | --//-- | --//-- | --//-- | --//-- | --//-- | “E27” |
| “E50” | --//-- | --//-- | --//-- | --//-- | “E19” | --//-- | --//-- | “E26” |
| “E51” | --//-- | --//-- | --//-- | --//-- | --//-- | --//-- | --//-- | “E27” |
| “E52” | --//-- | --//-- | “E8” | “E13” | “E18” | --//-- | --//-- | “E26” |
| “E53” | --//-- | --//-- | --//-- | --//-- | --//-- | --//-- | --//-- | “E27” |
| “E54” | --//-- | --//-- | --//-- | --//-- | “E19” | --//-- | --//-- | “E26” |
| “E55” | --//-- | --//-- | --//-- | --//-- | --//-- | --//-- | --//-- | “E27” |
| “E56” | --//-- | --//-- | “E9” | --//-- | “E18” | --//-- | --//-- | “E26” |
| “E57” | --//-- | --//-- | --//-- | --//-- | --//-- | --//-- | --//-- | “E27” |
| “E58” | --//-- | --//-- | --//-- | --//-- | “E19” | --//-- | --//-- | “E26” |
| “E59” | --//-- | --//-- | --//-- | --//-- | --//-- | --//-- | --//-- | “E27” |
| “E60” | --//-- | --//-- | “E8” | “E14” | “E18” | --//-- | --//-- | “E26” |
| “E61” | --//-- | --//-- | --//-- | --//-- | --//-- | --//-- | --//-- | “E27” |
| “E62” | --//-- | --//-- | --//-- | --//-- | “E19” | --//-- | --//-- | “E26” |
| “E63” | --//-- | --//-- | --//-- | --//-- | --//-- | --//-- | --//-- | “E27” |
| “E64” | --//-- | --//-- | “E9” | --//-- | “E18” | --//-- | --//-- | “E26” |
| “E65” | --//-- | --//-- | --//-- | --//-- | --//-- | --//-- | --//-- | “E27” |
| “E66” | --//-- | --//-- | --//-- | --//-- | “E19” | --//-- | --//-- | “E26” |
| “E67” | --//-- | --//-- | --//-- | --//-- | --//-- | --//-- | --//-- | “E27” |
| “E68” | --//-- | --//-- | “E8” | “E15” | “E18” | --//-- | --//-- | “E26” |
| “E69” | --//-- | --//-- | --//-- | --//-- | --//-- | --//-- | --//-- | “E27” |
| “E70” | --//-- | --//-- | --//-- | --//-- | “E19” | --//-- | --//-- | “E26” |
| “E71” | --//-- | --//-- | --//-- | --//-- | --//-- | --//-- | --//-- | “E27” |
| “E72” | --//-- | --//-- | “E9” | --//-- | “E18” | --//-- | --//-- | “E26” |
| “E73” | --//-- | --//-- | --//-- | --//-- | --//-- | --//-- | --//-- | “E27” |
| “E74” | --//-- | --//-- | --//-- | --//-- | “E19” | --//-- | --//-- | “E26” |
| “E75” | --//-- | --//-- | --//-- | --//-- | --//-- | --//-- | --//-- | “E27” |
| “E76” | --//-- | --//-- | “E8” | “E16” | “E18” | --//-- | --//-- | “E26” |
| “E77” | --//-- | --//-- | --//-- | --//-- | --//-- | --//-- | --//-- | “E27” |
| “E78” | --//-- | --//-- | --//-- | --//-- | “E19” | --//-- | --//-- | “E26” |
| “E79” | --//-- | --//-- | --//-- | --//-- | --//-- | --//-- | --//-- | “E27” |
| “E80” | --//-- | --//-- | “E9” | --//-- | “E18” | --//-- | --//-- | “E26” |
| “E81” | --//-- | --//-- | --//-- | --//-- | --//-- | --//-- | --//-- | “E27” |
| “E82” | --//-- | --//-- | --//-- | --//-- | “E19” | --//-- | --//-- | “E26” |
| “E83” | --//-- | --//-- | --//-- | --//-- | --//-- | --//-- | --//-- | “E27” |

The symbol “—//—” in Table 1 means that the variable defined thereby (e.g., the variable being substituent X) assumes the same value as stated for that variable in the previous row.

By means further illustration, Table 2 lists several non-limiting embodiments of the compound having the formula (I) and pharmaceutically acceptable salts thereof particularly preferred in the invention.

| Emb. | X, Y | n | m | p | q | R¹ | R² | R⁵ | R³ | R^{4, 6, 7} | R⁸ | R⁹ |
|---|---|---|---|---|---|---|---|---|---|---|---|---|
| “E84” | —O— | 0 | 0 | 0 | 0 | —COC₉ | —COC₉ | —COC₉ | —PO₃H₂ | —H | —C(═O)OH | —H |
| “E85” | —O— | 0 | 0 | 0 | 0 | —COC₉ | —COC₇ | —COC₇ | —PO₃H₂ | —H | —C(═O)OH | —H |
| “E86” | —O— | 0 | 0 | 0 | 0 | —COC₇ | —COC₉ | —COC₇ | —PO₃H₂ | —H | —C(═O)OH | —H |
| “E87” | —O— | 0 | 0 | 0 | 0 | —COC₇ | —COC₇ | —COC₉ | —PO₃H₂ | —H | —C(═O)OH | —H |
| “E88” | —O— | 0 | 0 | 0 | 0 | —COC₉ | —COC₉ | —COC₇ | —PO₃H₂ | —H | —C(═O)OH | —H |
| “E89” | —O— | 0 | 0 | 0 | 0 | —COC₉ | —COC₇ | —COC₉ | —PO₃H₂ | —H | —C(═O)OH | —H |
| “E89” | —O— | 0 | 0 | 0 | 0 | —COC₇ | —COC₉ | —COC₉ | —PO₃H₂ | —H | —C(═O)OH | —H |
| “E90” | —O— | 0 | 0 | 0 | 0 | —COC₉ | —COC₁₁ | —COC₁₁ | —PO₃H₂ | —H | —C(═O)OH | —H |
| “E91” | —O— | 0 | 0 | 0 | 0 | —COC₁₁ | —COC₉ | —COC₁₁ | —PO₃H₂ | —H | —C(═O)OH | —H |
| “E92” | —O— | 0 | 0 | 0 | 0 | —COC₁₁ | —COC₁₁ | —COC₉ | —PO₃H₂ | —H | —C(═O)OH | —H |
| “E93” | —O— | 0 | 0 | 0 | 0 | —COC₉ | —COC₉ | —COC₁₁ | —PO₃H₂ | —H | —C(═O)OH | —H |
| “E94” | —O— | 0 | 0 | 0 | 0 | —COC₉ | —COC₁₁ | —COC₉ | —PO₃H₂ | —H | —C(═O)OH | —H |
| “E95” | —O— | 0 | 0 | 0 | 0 | —COC₁₁ | —COC₉ | —COC₉ | —PO₃H₂ | —H | —C(═O)OH | —H |
| “E96” | —O— | 0 | 0 | 0 | 0 | —COC₉ | —COC₁₃ | —COC₁₃ | —PO₃H₂ | —H | —C(═O)OH | —H |
| “E97” | —O— | 0 | 0 | 0 | 0 | —COC₁₃ | —COC₉ | —COC₁₃ | —PO₃H₂ | —H | —C(═O)OH | —H |
| “E98” | —O— | 0 | 0 | 0 | 0 | —COC₁₃ | —COC₁₃ | —COC₉ | —PO₃H₂ | —H | —C(═O)OH | —H |
| “E99” | —O— | 0 | 0 | 0 | 0 | —COC₉ | —COC₉ | —COC₁₃ | —PO₃H₂ | —H | —C(═O)OH | —H |
| “E100” | —O— | 0 | 0 | 0 | 0 | —COC₉ | —COC₁₃ | —COC₉ | —PO₃H₂ | —H | —C(═O)OH | —H |
| “E101” | —O— | 0 | 0 | 0 | 0 | —COC₁₃ | —COC₉ | —COC₉ | —PO₃H₂ | —H | —C(═O)OH | —H |

The symbol “—COC₇” in Table 2 depicts the radical —C(═O)(CH₂)₆CH₃; “—COC₉” depicts the radical —C(═O)(CH₂)₈CH₃; “—COC₁₁” depicts the radical —C(═O)(CH₂)₁₀CH₃; and “—COC₁₃” depicts the radical —C(═O)(CH₂)₁₂CH₃.

Particularly preferably, the compound having the formula (I) and pharmaceutically acceptable salts thereof are as defined in embodiment “E84” in Table 2, i.e., wherein each of the substituents R¹, R² and R⁵ is the radical —C(═O)(CH₂)₈CH₃.

Some of the herein specified compounds employed by the invention may possess asymmetric carbon atoms (optical centres) or double bonds; the racemates, enantiomers, diastereomers, and any individual isomers (e.g., in pure form or in admixture with each other) are all intended to be encompassed within the scope of the present invention.

In a particularly preferred embodiment (“E102”), the invention employs the compound having the formula (II):

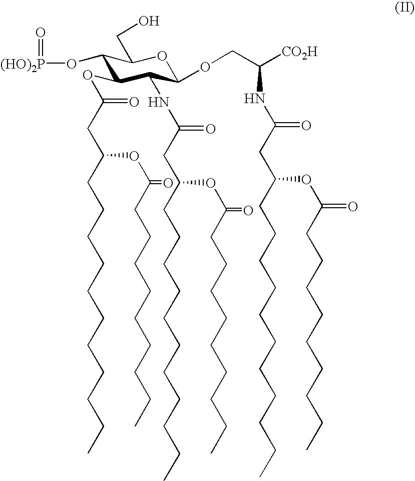

and pharmaceutically acceptable salts thereof.

The herein specified compounds may also exist in their tautomeric forms. Such forms, although not explicitly indicated in the above formulae are intended to be included within the scope of the present invention.

The above compounds may also contain unnatural proportions of atomic isotopes at one or more of the atoms that constitute such compounds. For example, the compounds may be radiolabelled with radioactive isotopes, such as for example tritium (³H), iodine-125 (¹²⁵I) or carbon-14 (¹⁴C). All isotopic variations of the compounds of the present invention, whether radioactive or not, are intended to be encompassed within the scope of the present invention.

The herein specified AGP compounds can be prepared by any suitable means, many of which have been described. For example, methods to prepare useful APG compounds have been described in U.S. Pat. No. 6,113,918, U.S. Pat. No. 6,303,347, WO 98/50300, WO 98/50399 and Johnson et al. 1999 (Bioorg Med Chem Lett 9: 2273-2278). In general, the synthetic methods described in the above-noted references and other synthetic methods otherwise familiar in the art are broadly applicable to the preparation these compounds. For example, in making compounds having different acyl groups and substitutions, one of skill in the art will appreciate that the convergent methods described therein can be modified to use alternate acylating agents, or can be initiated with commercially available materials having appropriate acyl groups attached.

The term “pharmaceutically acceptable” as used herein is consistent with the art and means compatible with the other ingredients of a pharmaceutical composition and not deleterious to the recipient thereof.

The term “pharmaceutically acceptable salts” is meant to include salts of the active compounds which are prepared with relatively non-toxic acids or bases, depending on the particular substituents found on the compounds described herein.

When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include alkali metal salts, such as, e.g., lithium, sodium or potassium salts, alkaline earth metal salts, such as, e.g., calcium or magnesium salts, and further aluminum salts, zinc salts, ammonium salts and organic amino salts, such as, e.g., tetramethylammonium salts, tetraethylammonium salts, salts with morpholine or piperidine, or salts with amino acids, such as, e.g., salts with lysine, arginine, glycine or phenylalanine, or the like bases.

Conversely, said base addition salt forms can be converted by treatment with an appropriate acid into the free acid form.

When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids, such as, e.g., hydrochloric, hydrobromic, nitric, carbonic, monohydrogencarbonic, phosphoric, monohydrogenphosphoric, dihydrogenphosphoric, sulphuric, monohydrogensulphuric, hydriodic or phosphorus acids or the like, as well as salts derived from relatively non-toxic organic acids, such as, e.g., acetic, hydroxyacetic, propionic, isobutyric, maleic, malonic, benzoic, succinic, suberic, fumaric, lactic, pyruvic, oxalic, malic, mandelic, phthalic, benzenesulfonic, p-tolylsulphonic, citric, tartaric, methanesulphonic, ethanesulphonic, salicylic, p-amino-salicylic, glucuronic or galactunoric acids or the like acids. See, e.g., Berge et al. (“Pharmaceutical Salts”, J Pharm Sci 66: 1-19).

Conversely said acid addition salt forms can be converted by treatment with an appropriate base into the free base form.

Certain specific compounds of the present invention contain both basic and acidic functionalities that allow the compounds to be converted into either base or acid addition salts.

Certain compounds of the present invention can exist in unsolvated forms as well as solvated forms, including hydrated forms, alcoholated forms and the like. In general, the solvated forms are equivalent to unsolvated forms and are intended to be encompassed within the scope of the present invention. Certain compounds of the present invention may exist in multiple crystalline or amorphous forms. In general, all physical forms are equivalent for the uses contemplated by the present invention and are intended to be within the scope of the present invention.

In addition to salt forms, the present invention also provides compounds which are in a prodrug form. Prodrugs of the compounds described herein are those compounds that readily undergo chemical changes under physiological conditions (in vivo) to provide the compounds of the present invention. Additionally, prodrugs can be converted to the compounds of the present invention by chemical or biochemical methods in an ex vivo environment. For example, prodrugs can be slowly converted to the compounds of the present invention when placed in a trans-dermal patch reservoir with a suitable enzyme or chemical reagent.

Hence, the term “prodrug” means the pharmacologically acceptable derivatives such as, e.g., esters, amides and phosphates, such that the resulting in vivo biotransformation product of the derivative is the active compounds as defined above. See, e.g., Goodman and Gilman (The Pharmacological Basis of Therapeutics, 8th ed, McGraw-Hill, Int. Ed. 1992, “Biotransformation of Drugs”, p 13-15) generally describing prodrugs. Prodrugs of a compound of the present invention are prepared by modifying functional groups present in the compound in such a way that the modifications are cleaved, either in routine manipulation or in vivo or ex vivo, to the parent compound. Prodrugs include compounds of the present invention wherein a hydroxy group or an amino group is bonded to any group that, when the prodrug is administered to a patient, cleaves to form a free hydroxyl or free amino, respectively.

Typical examples of prodrugs are described for instance in WO 99/33795, WO 99/33815, WO 99/33793 and WO 99/33792 all incorporated herein by reference. Prodrugs are usually characterised by excellent aqueous solubility, increased bioavailability and are readily metabolised into the active inhibitors in vivo.

Autoimmune Diseases

As explained, aspects of the invention provide prophylactic and therapeutic applications of the above defined compounds, and pharmaceutically acceptable salts or prodrugs thereof in autoimmune disorders.

As used herein, the term “autoimmune disease” or “autoimmune disorder” means a pathological condition, i.e., a disease or disorder, caused by an immune response against a self tissue or tissue component (self-antigen) and includes a self antibody response or cell-mediated response.

The term encompasses organ-specific autoimmune diseases, in which an autoimmune response is directed against a single tissue, as well as non-organ specific autoimmune diseases, in which an autoimmune response is directed against a component present in two or more, several or many organs throughout the body.

Specifically, in an embodiment, the autoimmune disease is chosen from the group comprising or consisting of: acute disseminated encephalomyelitis (ADEM); Addison's disease; ankylosing spondylitis; antiphospholipid antibody syndrome (APS); aplastic anemia; autoimmune gastritis; autoimmune hepatitis; autoimmune thrombocytopenia; Behçet's disease; coeliac disease; dermatomyositis; diabetes mellitus type I; Goodpasture's syndrome; Graves' disease; Guillain-Barré syndrome (GBS); Hashimoto's disease; idiopathic thrombocytopenic purpura; inflammatory bowel disease (IBD) including Crohn's disease and ulcerative colitis; mixed connective tissue disease; multiple sclerosis (MS); myasthenia gravis; opsoclonus myoclonus syndrome (OMS); optic neuritis; Ord's thyroiditis; pemphigus; pernicious anaemia; polyarteritis nodosa; polymyositis; primary biliary cirrhosis; primary myoxedema; psoriasis; rheumatic fever; rheumatoid arthritis; Reiter's syndrome; scleroderma; Sjögren's syndrome; systemic lupus erythematosus; Takayasu's arteritis; temporal arteritis; vitiligo; warm autoimmune hemolytic anemia; and Wegener's granulomatosis.

In a further embodiment, the autoimmune disease is preferably chosen from the group comprising or consisting of: autoimmune thrombocytopenia; diabetes mellitus type I; Graves' disease; Guillain-Barré syndrome (GBS); Hashimoto's disease; idiopathic thrombocytopenic purpura; inflammatory bowel disease (IBD) including Crohn's disease and ulcerative colitis; multiple sclerosis (MS); myasthenia gravis; psoriasis; rheumatoid arthritis; scleroderma; Sjögren's syndrome; systemic lupus erythematosus; and warm autoimmune hemolytic anemia.

In another particularly preferred embodiment, the autoimmune disease is chosen from the group comprising or consisting of: diabetes mellitus type I; Graves' disease; Guillain-Barré syndrome (GBS); Hashimoto's disease; inflammatory bowel disease (IBD) including Crohn's disease and ulcerative colitis; multiple sclerosis (MS); myasthenia gravis; psoriasis; rheumatoid arthritis; scleroderma; Sjögren's syndrome; and systemic lupus erythematosus.

In one preferred embodiment, the autoimmune disease is diabetes mellitus type I. In another preferred embodiment, the autoimmune disease is inflammatory bowel disease (IBD) including Crohn's disease and ulcerative colitis. In a further preferred embodiment, the autoimmune disease is myasthenia gravis. In yet another preferred embodiment, the autoimmune disease is psoriasis. In a further preferred embodiment, the autoimmune disease is rheumatoid arthritis. In still another preferred embodiment, the autoimmune disease is scleroderma. In yet another preferred embodiment, the autoimmune disease is systemic lupus erythematosus.

In a very preferred embodiment, the autoimmune disease is Guillain-Barré syndrome (GBS) or multiple sclerosis (MS). In a particularly preferred embodiment, the autoimmune disease is multiple sclerosis (MS).

Pharmaceutical Formulations

Compounds and pharmaceutically acceptable salts or prodrugs thereof as defined herein can be advantageously formulated as pharmaceutical formulations for treating autoimmune diseases.

Such pharmaceutical compositions comprise one or more compound or pharmaceutically acceptable salt or prodrug thereof (i.e., active substance) as defined herein, and one or more pharmaceutically acceptable carrier/excipient.

As used herein, “carrier” or “excipient” includes any and all solvents, diluents, buffers (such as, e.g., neutral buffered saline or phosphate buffered saline), solubilisers, colloids, dispersion media, vehicles, fillers, chelating agents (such as, e.g., EDTA or glutathione), amino acids (such as, e.g., glycine), proteins, disintegrants, binders, lubricants, wetting agents, emulsifiers, sweeteners, colorants, flavourings, aromatisers, thickeners, agents for achieving a depot effect, coatings, antibacterial and antifungal agents, preservatives, antioxidants, tonicity controlling agents, absorption delaying agents, and the like. The use of such media and agents for pharmaceutical active substances is well known in the art. Except insofar as any conventional media or agent is incompatible with the active substance, its use in the therapeutic compositions may be contemplated.

Illustrative, non-limiting carriers for use in formulating the pharmaceutical compositions include, for example, oil-in-water or water-in-oil emulsions, aqueous compositions with or without inclusion of organic co-solvents suitable for intravenous (IV) use, liposomes or surfactant-containing vesicles, microspheres, microbeads and microsomes, powders, tablets, capsules, suppositories, aqueous suspensions, aerosols, and other carriers apparent to one of ordinary skill in the art.

Said pharmaceutical compositions typically comprise a therapeutically effective amount of the one or more active substance, i.e., an amount which—under a suitable dosage regime—can elicit a biological or medicinal response in a tissue, system, animal or human that is being sought by a researcher, veterinarian, medical doctor or other clinician, and in particular can prevent or alleviate one or more of the local or systemic symptoms or features of the disease being treated.

Pharmaceutical compositions of the invention may be formulated for essentially any route of administration, such as without limitation, oral administration (such as, e.g., oral ingestion or inhalation), intranasal administration (such as, e.g., intranasal inhalation or intranasal mucosal application), parenteral administration (such as, e.g., subcutaneous, intravenous, intramuscular, intraperitoneal or intrasternal injection or infusion), transdermal or transmucosal (such as, e.g., oral, sublingual, intranasal) administration, rectal, vaginal or intra-tracheal instillation, and the like. In this way, the therapeutic effects attainable by the methods and compositions of the invention can be, for example, systemic, local, tissue-specific, etc., depending of the specific needs of a given application of the invention.

For example, for oral administration, pharmaceutical compositions may be formulated in the form of pills, tablets, lacquered tablets, coated (e.g., sugar-coated) tablets, granules, hard and soft gelatin capsules, aqueous, alcoholic or oily solutions, syrups, emulsions or suspensions. In an example, without limitation, preparation of oral dosage forms may be is suitably accomplished by uniformly and intimately blending together a suitable amount of the active compound in the form of a powder, optionally also including finely divided one or more solid carrier, and formulating the blend in a pill, tablet or a capsule. Exemplary but non-limiting solid carriers include calcium phosphate, magnesium stearate, talc, sugars (such as, e.g., glucose, mannose, lactose or sucrose), sugar alcohols (such as, e.g., mannitol), dextrin, starch, gelatin, cellulose, polyvinylpyrrolidine, low melting waxes and ion exchange resins. Compressed tablets containing the pharmaceutical composition can be prepared by uniformly and intimately mixing the active ingredient with a solid carrier such as described above to provide a mixture having the necessary compression properties, and then compacting the mixture in a suitable machine to the shape and size desired. Moulded tablets maybe made by moulding in a suitable machine, a mixture of powdered compound moistened with an inert liquid diluent. Suitable carriers for soft gelatin capsules and suppositories are, for example, fats, waxes, semisolid and liquid polyols, natural or hardened oils, etc.

For example, for oral or nasal aerosol or inhalation administration, pharmaceutical compositions may be formulated with illustrative carriers, such as, e.g., as in solution with saline, polyethylene glycol or glycols, DPPC, methylcellulose, or in mixture with powdered dispersing agents, further employing benzyl alcohol or other suitable preservatives, absorption promoters to enhance bioavailability, fluorocarbons, and/or other solubilising or dispersing agents known in the art. Suitable pharmaceutical formulations for administration in the form of aerosols or sprays are, for example, solutions, suspensions or emulsions of the compounds of the invention or their physiologically tolerable salts in a pharmaceutically acceptable solvent, such as ethanol or water, or a mixture of such solvents. If required, the formulation can also additionally contain other pharmaceutical auxiliaries such as surfactants, emulsifiers and stabilizers as well as a propellant. Illustratively, delivery may be by use of a single-use delivery device, a mist nebuliser, a breath-activated powder inhaler, an aerosol metered-dose inhaler (MDI) or any other of the numerous nebuliser delivery devices available in the art. Additionally, mist tents or direct administration through endotracheal tubes may also be used.

Examples of carriers for administration via mucosal surfaces depend upon the particular route, e.g., oral, sublingual, intranasal, etc. When administered orally, illustrative examples include pharmaceutical grades of mannitol, starch, lactose, magnesium stearate, sodium saccharide, cellulose, magnesium carbonate and the like, with mannitol being preferred. When administered intranasally, illustrative examples include polyethylene glycol, phospholipids, glycols and glycolipids, sucrose, and/or methylcellulose, powder suspensions with or without bulking agents such as lactose and preservatives such as benzalkonium chloride, EDTA. In a particularly illustrative embodiment, the phospholipid 1,2 dipalmitoyl-sn-glycero-3-phosphocholine (DPPC) is used as an isotonic aqueous carrier at about 0.01-0.2% for intranasal administration of the compound of the subject invention at a concentration of about 0.1 to 3.0 mg/ml.

For example, for parenteral administration, pharmaceutical compositions may be advantageously formulated as solutions, suspensions or emulsions with suitable solvents, diluents, solubilisers or emulsifiers, etc. Suitable solvents are, without limitation, water, physiological saline solution or alcohols, e.g. ethanol, propanol, glycerol, in addition also sugar solutions such as glucose, invert sugar, sucrose or mannitol solutions, or alternatively mixtures of the various solvents mentioned. The injectable solutions or suspensions may be formulated according to known art, using suitable non-toxic, parenterally-acceptable diluents or solvents, such as mannitol, 1,3-butanediol, water, Ringer's solution or isotonic sodium chloride solution, or suitable dispersing or wetting and suspending agents, such as sterile, bland, fixed oils, including synthetic mono- or diglycerides, and fatty acids, including oleic acid. The compounds and pharmaceutically acceptable salts thereof of the invention can also be lyophilised and the lyophilisates obtained used, for example, for the production of injection or infusion preparations. For example, one illustrative example of a carrier for intravenous use includes a mixture of 10% USP ethanol, 40% USP propylene glycol or polyethylene glycol 600 and the balance USP Water for Injection (WFI). Other illustrative carriers for intravenous use include 10% USP ethanol and USP WFI; 0.01-0.1% triethanolamine in USP WFI; or 0.01-0.2% dipalmitoyl diphosphatidylcholine in USP WFI; and 1-10% squalene or parenteral vegetable oil-in-water emulsion. Illustrative examples of carriers for subcutaneous or intramuscular use include phosphate buffered saline (PBS) solution, 5% dextrose in WFI and 0.01-0.1% triethanolamine in 5% dextrose or 0.9% sodium chloride in USP WFI, or a 1 to 2 or 1 to 4 mixture of 10% USP ethanol, 40% propylene glycol and the balance an acceptable isotonic solution such as 5% dextrose or 0.9% sodium chloride; or 0.01-0.2% dipalmitoyl diphosphatidylcholine in USP WFI and 1 to 10% squalene or parenteral vegetable oil-in-water emulsions.

Where aqueous formulations are preferred, such may comprise one or more surfactants. For example, the composition can be in the form of a micellar dispersion comprising at least one suitable surfactant, e.g., a phospholipid surfactant. Illustrative examples of phospholipids include diacyl phosphatidyl glycerols, such as dimyristoyl phosphatidyl glycerol (DPMG), dipalmitoyl phosphatidyl glycerol (DPPG), and distearoyl phosphatidyl glycerol (DSPG), diacyl phosphatidyl cholines, such as dimyristoyl phosphatidylcholine (DPMC), dipalmitoyl phosphatidylcholine (DPPC), and distearoyl phosphatidylcholine (DSPC); diacyl phosphatidic acids, such as dimyristoyl phosphatidic acid (DPMA), dipahnitoyl phosphatidic acid (DPPA), and distearoyl phosphatidic acid (DSPA); and diacyl phosphatidyl ethanolamines such as dimyristoyl phosphatidyl ethanolamine (DPME), dipalmitoyl phosphatidyl ethanolamine (DPPE) and distearoyl phosphatidyl ethanolamine (DSPE). Typically, a surfactant:active substance molar ratio in an aqueous formulation will be from about 10:1 to about 1:10, more typically from about 5:1 to about 1:5, however any effective amount of surfactant may be used in an aqueous formulation to best suit the specific objectives of interest.

When rectally administered in the form of suppositories, these formulations may be prepared by mixing the compounds according to the invention with a suitable non-irritating excipient, such as cocoa butter, synthetic glyceride esters or polyethylene glycols, which are solid at ordinary temperatures, but liquidify and/or dissolve in the rectal cavity to release the drug.

Suitable carriers for microcapsules, implants or rods are, for example, copolymers of glycolic acid and lactic acid.

One skilled in this art will recognize that the above description is illustrative rather than exhaustive. Indeed, many additional formulations techniques and pharmaceutically-acceptable excipients and carrier solutions are well-known to those skilled in the art, as is the development of suitable dosing and treatment regimens for using the particular compositions described herein in a variety of treatment regimens.

In a further modality, a pharmaceutical composition of the invention may comprise, in addition to the compound having formula (I) or pharmaceutically acceptable salt or prodrug thereof as defined above (i.e., the active substance according to the invention), also one or more other active compounds that are suitable in the treatment of an autoimmune disease.

Treatment

As noted, the invention also concerns the treatment of autoimmune diseases in subjects needing such therapy, comprising administering a therapeutically effective amount of one or more above active substances of the invention, preferably as suitable pharmaceutical compositions, to said subjects.

“Subject” or “patient” as used herein refer to animals, preferably vertebrates, more preferably mammals, and specifically includes human patients and non-human mammal subjects. The term “mammal” includes any animal classified as such, including, but not limited to, humans, domestic and farm animals, zoo animals, sport animals, pet animals, companion animals and experimental animals, such as, for example, mice, rats, hamsters, rabbits, dogs, cats, guinea pigs, cattle, cows, sheep, horses, pigs and primates, e.g., monkeys and apes.

Preferred patients are human subjects.

As used herein, the terms “treat” or “treatment” refer to both therapeutic treatment and prophylactic or preventative measures, wherein the object is to prevent or slow down (lessen) an undesired physiological change or disorder, such as the development or progression of an autoimmune disorder. Beneficial or desired clinical results include, but are not limited to, alleviation of one or more symptoms or one or more biological markers (e.g., level of auto-antibodies), diminishment of extent of disease, stabilised (i.e., not worsening) state of disease, delay or slowing of disease progression, amelioration or palliation of the disease state, prolongation of time between relapses, etc. “Treatment” can also mean prolonging survival as compared to expected survival if not receiving treatment.

As used herein, a phrase such as “a subject in need of treatment” includes subjects, such as mammalian subjects, more preferably human subjects, that would benefit from treatment of a given condition, preferably an autoimmune disease. Such subjects will typically include, without limitation, those that have been diagnosed with the condition, those prone to have or develop the said condition and/or those in whom the condition is to be prevented.

The active substances of the invention may be used alone or in combination with any of the autoimmune disease therapies known in the art, such as, e.g., anti-inflammatory agents, e.g., interferon beta-1a or beta-1b. Said additional anti-autoimmune disease agents can be administered before, after or simultaneously with the administration of the active substances according to the invention.

The dosage or amount of active substances of the invention used, optionally in combination with one or more other active compound to be administered, depends on the individual case and is, as is customary, to be adapted to the individual circumstances to achieve an optimum effect. Thus, it depends on the nature and the severity of the disorder to be treated, and also on the sex, age, body weight, general health, diet, mode and time of administration, and individual responsiveness of the human or animal to be treated, on the route of administration, efficacy, metabolic stability and duration of action of the compounds used, on whether the therapy is acute or chronic or prophylactic, or on whether other active compounds are administered in addition to the agent(s) of the invention.

Without limitation, depending on the type and severity of the disease, a typical daily dosage might range from about 1 μg/kg to 100 mg/kg of body weight or more, depending on the factors mentioned above. For repeated administrations over several days or longer, depending on the condition, the treatment is sustained until a desired suppression of disease symptoms occurs. A preferred dosage of the active substance of the invention may be in the range from about 0.05 mg/kg to about 10 mg/kg of body weight. Thus, one or more doses of about 0.5 mg/kg, 2.0 mg/kg, 4.0 mg/kg or 10 mg/kg (or any combination thereof) may be administered to the patient. Such doses may be administered intermittently, e.g., every week or every three weeks.

The above aspects and embodiments are further supported by the following examples which are in no instance to be considered limiting.

### Examples

Improved Condition of EAE Mice by Compound of Formula (II)

Experimental autoimmune encephalomyelitis (EAE) is a demyelinating disease of the central nervous system in mice and rats widely used as a model of multiple sclerosis (MS). The disease appears in exacerbations and remissions and is characterised by loss of nerve conduction and chronic progression of disability. Macrophages and T-lymphocytes appear to mediate the destruction of the myelin sheath surrounding the nerves, resulting in improper nerve conduction. Research into the pathogenesis of EAE has shown that it may display many similarities to other autoimmune and inflammatory diseases.

EAE can be induced by immunisation of experimental animals with well-defined myelin-derived antigens including myelin basic protein (MBP) and proteolipid protein (PLP) antigens (Martin et al. 1992, “Immunological aspects of demyelinating diseases”, Annu Rev Immunol 10: 153-87). For example, SJL mice can be immunised with myelin-derived peptides PLP₁₃₉₋₁₅₁, and C57BL/6 mice with peptide MOG₃₅₋₅₅, or DA rats with MOG₁₋₁₂₅ or Lewis rats with MBP₆₃₋₈₈.

In the most frequently used SJL mouse model, first clinical symptoms appear about 10 days post-immunisation. The first phase of clinical activity is followed by a relapse in a majority of the sensitised animals. Clinical parameters of disease that can be monitored in the model include, e.g., loss of bodyweight and paralysis/paresis. The duration of the monitoring period depends on whether the primary interest is interference with the first phase of disease, or interference with the chronic relapsing phase. The endpoint of the experiment is the analysis of the extent of inflammation in both brain tissue and spinal cord.

In the present experiment, female SJL/J mice are immunised with 100 μg synthetic peptide comprising amino acids 139-151 from PLP (PLP₁₃₉₋₁₅₁). Development of EAE is established by assessment of body weight and the extent of paralysis/paresis (of tail, hind limbs and/or general), and post mortem histology of CNS.

Half of the mice is administered the compound of formula (II) in a suitable solvent, such as PBS, while the other half is administered solvent only. The mice treated with the compound of formula (II) show improvement of the clinical picture (higher body weight, less paralysis/paresis), and show less CNS inflammation, compared to the control mice.

IL-17 Response in EAE Mice to Compound of Formula (II)

IL-17 produced by autoreactive CD4 positive T cells belonging to the Th17 subset plays a key role in several autoimmune diseases such as multiple sclerosis, Crohn's disease, rheumatoid arthritis and psoriasis (Batten et al. 2006. Nat Immunol 7: 929-36; Park et al. 2005. Nat Immunol 6: 1133-41; Afzali et al. 2007. Clin Exp Immunol 148: 32-46; Furuzawa-Carballeda et al. 2007. Autoimmun Rev. 6: 169-75).

We determined the impact of compound of formula (II) administration on the emergence of autoreactive IL-17-secreting cells upon immunization with a myelin-derived proteolipid peptide, which induces experimental autoimmune encephalomyelitis, an animal model of multiple sclerosis.

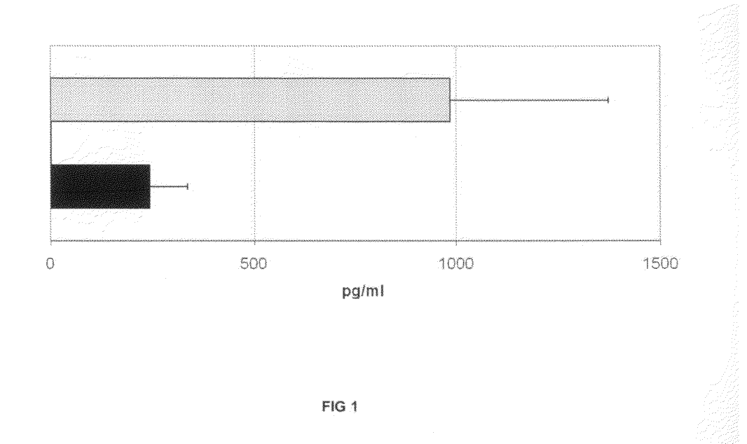

SJL mice were injected with the PLP₁₃₉₋₁₅₁ peptide which is well known as a potent inducer of the illness. Afterwards, mice were treated with compound of formula (II) at 2.5 μg/mouse. Cells from the draining lymph nodes were then harvested and stimulated in vivo with the antigenic peptide. Supernatants were harvested and assessed for their IL-17 concentration. Cells extracted from mice treated with compound of formula (II) after the induction of the disease display significant reduction in the production of IL-17 (p<0.05; see FIG. 1). This shows that the compound has a beneficial effect on pathologies related to IL-17 cytokine, including in particular multiple sclerosis, rheumatoid arthritis, psoriasis and Crohn's disease.

1. Compound having the formula (I): 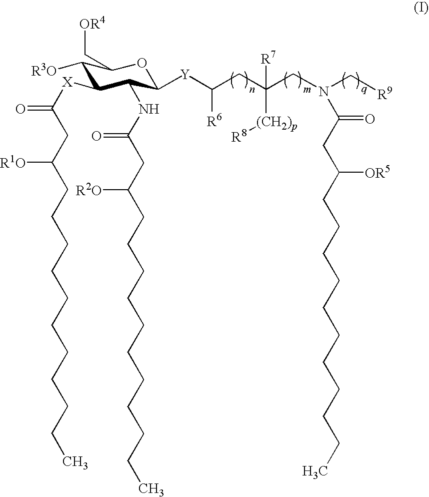
and pharmaceutically acceptable salts thereof, wherein:
X is —O— or —NH—; Y is —O— or —S—; the subscripts n, m, p and q are each independently an integer of from O to 6; R¹, R² and R⁵ are each independently a (C₈-C₁₄)acyl group and at least one of R¹, R² and R⁵ is a (C₁₀)acyl group; R³ is —H or —PO₃R¹¹R¹², wherein R¹¹ and R¹² are each independently —H or (C₁-C₄)alkyl; R⁴ is —H, —CH₃ or PO₃R¹³R¹⁴, wherein R¹³ and R¹⁴ are each independently —H or (C₁-C₄)alkyl, with the proviso that when R³ is —PO₃R¹¹R¹², R⁴ is other than —PO₃R¹³R¹⁴; R⁶ and R⁷ are each independently —H or —CH₃; and R⁸ and R⁹ are each independently —H, —OH, (C₁-C₄)alkoxy, —PO₃R¹⁵R¹⁶, —OPO₃R¹⁵R¹⁶, —SO₃R¹⁵, —OSO₃R¹⁵, —NR¹⁵R¹⁶, —SR¹⁵, —C≡N, —NO₂, —C(═O)H, —C(═O)OR¹⁵, or —C(═O)NR¹⁵R¹⁶, wherein R¹⁵ and R¹⁶ are each independently —H or (C₁-C₄)alkyl; for use in the treatment of an autoimmune disease.

2. The compound according to claim 1, wherein at least two of R¹, R² and R⁵ are a (C₁₀)acyl group.

3. The compound according to claim 1, wherein all three R¹, R² and R⁵ are a (C₁₀)acyl group.

4. The compound according to claim 1, wherein at least one, more preferably at least two, and most preferably all three of said (C₁₀)acyl groups are straight and/or un-substituted and/or saturated (C₁₀)acyl groups, preferably straight and un-substituted and saturated (C₁₀)acyl groups.

5. The compound according to claim 1, wherein X is —O—; Y is —O—; the subscripts n, m, p, q are each 0; R³ is —PO₃H₂; R⁴, R⁶, R⁷ and R⁹ are each —H; and R⁸ is —C(═O)OH.

6. The compound according to claim 1, having the formula (II):

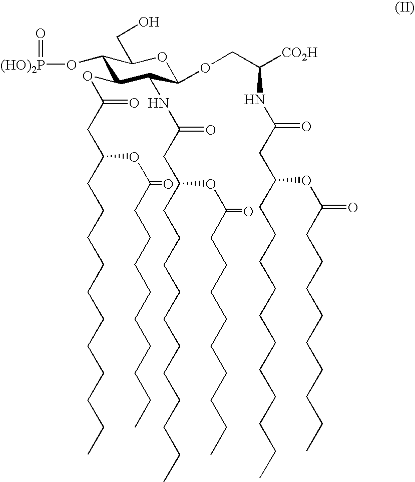

7. The method according to claim 12, wherein the autoimmune disease is selected from the group consisting of: acute disseminated encephalomyelitis (ADEM); Addison's disease; ankylosing spondylitis; antiphospholipid antibody syndrome (APS); aplastic anemia; autoimmune gastritis; autoimmune hepatitis; autoimmune thrombocytopenia; Behcet's disease; coeliac disease; dermatomyositis; diabetes mellitus type I; Goodpasture's syndrome; Graves' disease; Guillain-Barré syndrome (GBS); Hashimoto's disease; idiopathic thrombocytopenic purpura; inflammatory bowel disease (IBD) including Crohn's disease and ulcerative colitis; mixed connective tissue disease; multiple sclerosis (MS); myasthenia gravis; opsoclonus myoclonus syndrome (OMS); optic neuritis; Ord's thyroiditis; pemphigus; pernicious anaemia; polyarteritis nodosa; polymyositis; primary biliary cirrhosis; primary myoxedema; psoriasis; rheumatic fever; rheumatoid arthritis; Reiter's syndrome; scleroderma; Sjogren's syndrome; systemic lupus erythematosus; Takayasu's arteritis; temporal arteritis; vitiligo; wain' autoimmune hemolytic anemia; and Wegener's granulomatosis.

8. The method according to claim 12, wherein the autoimmune disease is selected from the group consisting of: autoimmune thrombocytopenia; diabetes mellitus type I; Graves' disease; Guillain-Barré syndrome (GBS); Hashimoto's disease; idiopathic thrombocytopenic purpura; inflammatory bowel disease (IBD) including Crohn's disease and ulcerative colitis; multiple sclerosis (MS); myasthenia gravis; psoriasis; rheumatoid arthritis; scleroderma; Sjogren's syndrome; systemic lupus erythematosus; and warm autoimmune hemolytic anemia.

9. The method according to claim 12, wherein the autoimmune disease is multiple sclerosis (MS).

10. A medicament for the treatment of an autoimmune disease comprising the compound of claim 1 in a pharmaceutically acceptable carrier.

11. The medicament according to claim 10, wherein the autoimmune disease is selected from the group consisting of: acute disseminated encephalomyelitis (ADEM); Addison's disease; ankylosing spondylitis; antiphospholipid antibody syndrome (APS); aplastic anemia; autoimmune gastritis; autoimmune hepatitis; autoimmune thrombocytopenia; Behcet's disease; coeliac disease; dermatomyositis; diabetes mellitus type I; Goodpasture's syndrome; Graves' disease; Guillain-Barré syndrome (GBS); Hashimoto's disease; idiopathic thrombocytopenic purpura; inflammatory bowel disease (IBD) including Crohn's disease and ulcerative colitis; mixed connective tissue disease; multiple sclerosis (MS); myasthenia gravis; opsoclonus myoclonus syndrome (OMS); optic neuritis; Ord's thyroiditis; pemphigus; pernicious anaemia; polyarteritis nodosa; polymyositis; primary biliary cirrhosis; primary myoxedema; psoriasis; rheumatic fever; rheumatoid arthritis; Reiter's syndrome; scleroderma; Sjogren's syndrome; systemic lupus erythematosus; Takayasu's arteritis; temporal arteritis; vitiligo; warm autoimmune hemolytic anemia; and Wegener's granulomatosis.

12. A method of treating an autoimmune disease comprising administering the compound having the formula (I) or pharmaceutically acceptable salts thereof to an individual in need thereof, 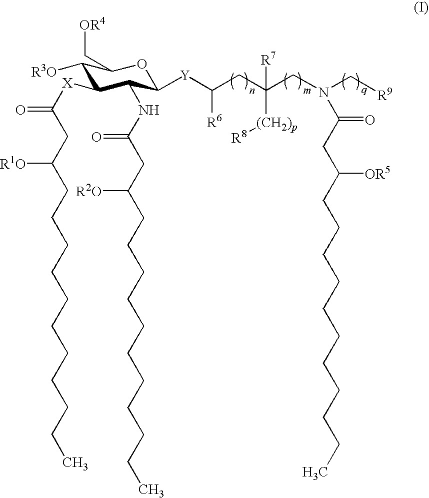
wherein:
X is —O— or —NH—; Y is —O— or —S—; the subscripts n, m, p and q are each independently an integer of from O to 6; R¹, R² and R⁵ are each independently a (C₈-C₁₄)acyl group and at least one of R¹, R² and R⁵ is a (C₁₀)acyl group; R³ is —H or —PO₃R¹¹R¹², wherein R¹¹ and R¹² are each independently —H or (C₁-C₄)alkyl; R⁴ is —H, —CH₃ or PO₃R¹³R¹⁴, wherein R¹³ and R¹⁴ are each independently —H or (C₁-C₄)alkyl, with the proviso that when R³ is —PO₃R¹¹R¹², R⁴ is other than —PO₃R¹³R¹⁴; R⁶ and R⁷ are each independently —H or —CH₃; and R⁸ and R⁹ are each independently —H, —OH, (C₁-C₄)alkoxy, —PO₃R¹⁵R¹⁶, —OPO₃R¹⁵R¹⁶, —SO₃R¹⁵, —OSO₃R¹⁵, —NR¹⁵R¹⁶, —SR¹⁵, —C≡N, —NO₂, —C(═O)H, —C(═O)OR¹⁵, or —C(═O)NR¹⁵R¹⁶, wherein R¹⁵ and R¹⁶ are each independently —H or (C₁-C₄)alkyl.

13. The method according to claim 12, wherein at least two of R¹, R² and R⁵ are a (C₁₀)acyl group in the compound of formula (I).

14. The method of claim 12, wherein all three R¹, R² and R⁵ are a (C₁₀)acyl group in the compound of formula (I).

15. The method according to claim 12, wherein at least one, more preferably at least two, and most preferably all three of said (C₁₀)acyl groups are straight and/or un-substituted and/or saturated (C₁₀)acyl groups, preferably straight and un-substituted and saturated (C₁₀)acyl groups in the compound of formula (I).

16. The method according to claim 12, wherein X is —O—; Y is —O—; the subscripts n, m, p, q are each 0; R³ is —PO₃H₂; R⁴, R⁶, R⁷ and R⁹ are each —H; and R⁸ is —C(═O)OH in the compound of formula (I).

17. The method according to claim 12, wherein the compound is represented by formula (II): [chemical structure]
